(19) United States
(12) Patent Application Publication
(10) Pub. No.: US 2026/0055159 A1
(43) Pub. Date: Feb. 26, 2026

(54) COMBINATION OF NY-ESO-1 SPECIFIC T CELL RECEPTORS AND CHIMERIC CO-STIMULATORY RECEPTORS

(71) Applicant: MEDIGENE IMMUNOTHERAPIES GMBH, Planegg-Martinsried (DE)

(72) Inventors: Maja BÜRDEK, München (DE); Kathrin MUTZE, München (DE); Petra PRINZ, Gilching (DE); Angelika HELMBRECHT, Neufarn (DE)

(21) Appl. No.: 19/105,413

(22) PCT Filed: Apr. 3, 2023
(86) PCT No.: PCT/EP2023/058651

§ 371 Date: Feb. 21, 2025

(30) Foreign Application Priority Data
Aug. 23, 2022 (WO) PCT/EP2022/073443

(51) Int. Cl. C07K 14/725; A61K 35/17; A61K 40/11; A61K 40/32; A61K 40/42; A61P 35/00; C07K 14/705; C07K 14/715; C12N 5/0783

(52) U.S. Cl. CPC C07K 14/7051; A61K 35/17; A61K 40/11; A61K 40/32; A61K 40/4269; A61P 35/00; C07K 14/70596; C07K 14/7151; C12N 5/0636; C07K 2317/565; C07K 2319/03

(57) ABSTRACT

The present invention refers immune cells expressing a TCR and a co-stimulatory. In particular, the invention refers to immune cells expressing a (i) T cell receptor (TCR) specific for a TCR specific for NY-ESO-1 peptide SLLMWITQC and (ii) a chimeric co-stimulatory receptor comprising an extracellular domain derived from PD-1 (CD279) and an intracellular domain derived from 4-1BB (CD137).

### FIELD OF THE INVENTION

The present invention refers immune cells expressing a TCR and a co-stimulatory. In particular, the invention refers to immune cells expressing a (i) T cell receptor (TCR) specific for a TCR specific for NY-ESO-1 peptide SLLMWITQC and (ii) a chimeric co-stimulatory receptor comprising an extracellular domain derived from PD-1 (CD279) and an intracellular domain derived from 4-1BB (CD137).

### BACKGROUND OF THE INVENTION

NY-ESO-1 and LAGE-1 are important immunotherapeutic target antigens belonging to the family of Cancer/Testis antigens. Cancer/Testis antigens are expressed in various malignant tumors and germ cells of the testis but not on other adult tissues.

Success of immunotherapies with TCR-modified T cells depends not only on the choice of a target antigen but also the selection of a TCR with high antigen specificity and sensitivity. An additional challenge, particularly in treatment of solid tumors, is the immunosuppressive tumor microenvironment (TME) that negatively influences efficacy, fitness and persistence of TCR-modified T cells. In addition to inhibitory cytokines and deprivation of essential metabolic factors, T cells face the inhibitory checkpoint PD-1/PD-L1 axis in the TME that reduces T cell infiltration and causes their exhaustion. Consequently, new strategies are needed to equip TCR-modified T cells with traits to overcome an inhibitory immunosuppressive TME. More specifically, the TCR-modified T cells targeting specific antigens, such as NY-ESO-1 with high specificity and with enhanced proliferation, cytokine release and cytotoxicity are desired.

### OBJECTIVES AND SUMMARY OF THE INVENTION

To overcome these needs, the present invention provides a combination of a high avidity TCR and a chimeric co-stimulatory receptor allowing the generation of highly specific T cells targeting antigens, for example NY-ESO-1, with enhanced cytokine release, proliferation and cytotoxicity, in particular polyfunctional immune cells secreting 2 or more cytokines.

The invention refers to a target specific immune cell expressing
(A) an antigen specific TCR, and
(B) a chimeric co-stimulatory receptor,

wherein the target specific immune cell secretes at least two proteins.

Thus, one embodiment of the present invention is the provision of a cell comprising
(A) a NY-ESO-1/LAGE-1 specific TCR, comprising
   a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
   a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40.
(B) a chimeric co-stimulatory receptor comprising
   an extracellular domain containing a polypeptide derived from PD-1,
   a transmembrane domain, and
   an intracellular domain containing a polypeptide derived from 4-1BB,

The NY-ESO-1-specific TCR used is capable of binding to a NY-ESO-1 peptide having the amino acid sequence SLLMWITQC (SEQ ID NO: 34) or a portion thereof, or its HLA-A2 bound form. It provides high functional avidity and advantageous tumor cell recognition and killing properties. The co-stimulatory receptor reverses the inhibitory checkpoint axis PD-1/PD-L1 to improve the T cell functionality, in particular in a suppressive TME. Thus, the combination of the inventive TCR and the chimeric co-stimulatory receptor allows improved targeting of NY-ESO-1 with high specificity and with enhanced proliferation, cytokine release and cytotoxicity.

The chimeric co-stimulatory receptor may comprise a transmembrane domain which is derived from PD-1. In specific embodiments the sequence of chimeric co-stimulatory receptor may comprise the sequence of SEQ ID NO: 26.

Accordingly, a further aspect relates to a composition comprising
a nucleic acid encoding a NY-ESO-1 specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, one aspect relates to a nucleic acid comprising
a nucleic acid encoding a NY-ESO-1 specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Another aspect refers to a vector comprising the nucleic acid comprising the sequences for the NY-ESO-1-specific TCR and the chimeric co-stimulatory receptor. Also cells comprising the nucleic acid composition and/or the vector are encompassed.

Typically, the cell is a peripheral blood lymphocyte (PBL) or a peripheral blood mononuclear cell (PBMC). In a specific embodiment, the cell is a T cell.

### Polyfunctional Immune Cells

One objective of the invention refers to a cell population comprising cells expressing
(A) an antigen specific TCR
(B) a chimeric co-stimulatory receptor

wherein the cell population comprises cells which secrete at least two proteins.

In some embodiments, the chimeric co-stimulatory receptor comprises
an extracellular domain containing a polypeptide derived from PD-1,
a transmembrane domain derived from PD-1, and
an intracellular domain containing a polypeptide derived from 4-1BB,

wherein the cell population comprises cells which secrete at least two proteins.

A further aspect of the invention refers to a target specific immune cell expressing
(A) an antigen specific TCR, and
(B) a chimeric co-stimulatory receptor as described herein,

wherein the target specific immune cell secretes at least two proteins.

In some embodiments the cells secrete at least three proteins, such as at least four or at least 5 proteins.

Further aspects refer to a pharmaceutical composition comprising the cell, the composition, the nucleic acid and the vector defined herein. Further aspects refer to the cell, the composition, the nucleic acid and the vector defined herein for cancer treatment.

Surprisingly the inventors found that TCR-T cells expressing chimeric-costimulatory receptor as defined herein show a higher polyfunctionality compared to TCR-T cells lacking chimeric-costimulatory receptor as defined herein. Higher polyfunctionality of transgenic T cells point to a higher functionality and anti-tumor activity in vivo and correlates with clinical outcome.

In particular we could show that a cell population expressing both the target specific NY-ESO-2 specific TCR and the chimeric-costimulatory receptor as defined herein comprises cells which secrete at least two proteins, e.g. at least three proteins, at least four proteins, at least 5 proteins, at least 6 proteins.

### FIGURE LEGENDS

FIG. 1: TCR-T cells targeting NY-ESO-1 expressing PD1-41BB show a higher polyfunctionality compared to TCR-T cells targeting NY-ESO-1 lacking PD1-41BB. TCR-transgenic T cells with or without PD1-41BB were analyzed regarding their single-cell polyfunctionality (release of 2 or more cytokines) using the IsoLight® technology (IsoPlexis). After co-culture with PD-L1-overexpressing, NY-ESO-1 positive MelA375 and Mel624.38 tumor cells, single CD8+ T cells were evaluated for the secretion of 32 T cell cytokines/proteins and compared with untransduced TCR-T cells.
(A) TCR-T cells expressing PD1-41BB showed a higher percentage of poly-functional T cells compared to TCR-T cells lacking PD1-41BB. Different shades of grey show how many cytokines were released by a single T cell simultaneously.
(B) The poly-functional strength index (PSI) was calculated by multiplying the intensities of the various secreted cytokines and the percentage of polyfunctional T cells. TCR-T cells expressing PD1-41BB showed a higher polyfunctional strength index (PSI) compared to TCR-T cells lacking PD1-41BB. Classification of the various cytokines/lytic proteins released revealed a high contribution of effector (Gzm-B, IFN-γ, MIP-1a, Perforin, TNF-α, TNF-β) and stimulatory (GM-CSF, IL-2, IL-5, IL-8, IL-9, IL-12) cytokines/lytic proteins to the superior PSI, followed by chemo-attractive (IP-10, MIP-1β, RANTES) cytokines. Regulatory (IL-4, IL-10, IL-22, sCD137, TGF-β1) and inflammatory (IL-6, IL-17F, MCP-1) cytokines were released to a lower extent.
(C) Detailed analysis of the single-cell poly-cytokine release using a polyfunctionality heat map showed that TCR-T cells with and without PD1-41BB differed in their poly-cytokine signature. Notably, TCR-T cells expressing PD1-41BB contained a higher proportion of single cells secreting 2-6 cytokines simultaneously.

FIG. 2: Improved effector function of NY-ESO-1 TCR-T cells co-expressing chimeric PD1-41BB co-stimulatory receptor. (A) Flow cytometric analysis of untransduced (UT), NY-ESO-1 TCR expressing (TCR) and NY-ESO-1 TCR co-expressing PD1-41BB (TCR+PD1-41BB) T cells. (B) NY-ESO-1 TCR transgenic CD8+ T cells with or without PD1-41BB were cultured with tumor cell lines expressing different levels of NY-ESO-1 and PD-L1 and released IFN-7 levels were determined by ELISA (t-test, **p>0.01, ***p<0.001). (C) Expression levels of NY-ESO-1 were measured by qPCR and quantified against a standard calibration curve. The levels of PD-L1 summarized here were evaluated by both qPCR and FACS analysis. (D) NY-ESO-1 TCR transgenic CD8+ T cells with or without PD1-41BB were stained with the dye membrane tracker and co-cultured with NY-ESO-1+PD-L1+ tumor cell lines for 5 days. Proliferation was analyzed via flow cytometry and data were evaluated using FlowJo.

FIG. 3: Enhanced NY-ESO-1 TCR-T cell polyfunctionality by co-expression of chimeric co-stimulatory receptor PD1-41BB. NY-ESO-1 specific TCR-T cells with (TCR+PD1-41BB) and without (TCR) co-expression of chimeric co-stimulatory receptor PD1-41BB were analyzed after 24 hours of co-culture with tumor target cell lines Mel624.38_PD-L1 and MelA375_PD-L1 using single cell proteomic analysis of a panel of 32 secreted cytokines, chemokines, and cytotoxic molecules (IsoLight technology, IsoPlexis). Co-culture with un-transduced T cells (UT) of the same donor served as control. Cells secreting 2 or more cytokines are considered polyfunctional. (A) Polyfunctionality of single TCR T cells displayed as % of sample and categorized in % cells expressing 2, 3, 4 and 5+ analytes (shades of orange) at the same time. (B) Polyfunctional Strength Index (PSI) of the displayed samples is defined as the number of T cells secreting more than 2 effector molecules per cell (polyfunctional T cells in a)), multiplied by mean fluorescence intensity (MFI) of the proteins secreted by the respective cells and categorized in proteins associated with effector, stimulatory, chemoattractive, regulatory and inflammatory properties (shades of grey). (C) Single-cell polyfunctional heat map displays the single-cell cytokine combinations secreted by each sample. Each column represents a specific combination of cytokines, whereas the orange squares depict the frequency at which the specific combination of cytokines is secreted by the corresponding sample (average for target cells Mel624.38_PD-L1 and MelA375_PD-L1 is shown). The cytokine groups are ordered by polyfunctionality and only the 40 most polyfunctional groups present within the samples are shown.

FIG. 4: Increased frequencies of secreted effector, stimulatory, and chemoattractive proteins by TCR-T cells co-expressing co-stimulatory receptor PD1-41BB. Secretion frequency of single proteins induced by co-culture of TCR T cells equipped with PD1-41BB cells and target cell line Mel624.38_PD-L1. Proteins are displayed and categorized by function due to their a) effector, b) stimulatory, c) chemoattractive, d) regulatory and e) inflammatory properties.

FIG. 5: Serial killing of tumor cell spheroids. Tumor cell spheroids were generated starting with 1000 NucLightRed-transduced cells in 96 well ULA plates 72 h before co-culture. TCR-transduced T cells with and without PD1-41BB (10,000-38,000 per well) were added and tumor cell killing was monitored by real-time imaging of NucLightRed signal using the IncuCyte S3. Fresh tumor cell spheroids, generated as described above, were added every 72-96 h.

### DETAILED DESCRIPTION OF THE INVENTION

Before the invention is described in detail with respect to some of its preferred embodiments, the following general definitions are provided.

The present invention as illustratively described in the following may be suitably practiced in the absence of any element or elements, limitation or limitations, not specifically disclosed herein.

The present invention will be described with respect to particular embodiments and with reference to certain figures, but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense or meaning to the person skilled in the art. If a specific meaning is conveyed to certain terms, definitions of terms will be given in the following in the context of which the terms are used.

### TCR Background

A TCR is composed of two different and separate protein chains, namely the TCR alpha (α) and the TCR beta (β) chain. The TCR α chain comprises variable (V), joining (J) and constant (C) regions. The TCR β chain comprises variable (V), diversity (D), joining (J) and constant (C) regions. The rearranged V(D)J regions of both the TCR α and the TCR β chain contain hypervariable regions (CDR, complementarity determining regions), among which the CDR3 region determines the specific epitope recognition. At the C-terminal region both TCR α chain and TCR β chain contain a hydrophobic transmembrane domain and end in a short cytoplasmic tail.

Typically, the TCR is a heterodimer of one α chain and one β chain. This heterodimer can bind to MHC molecules presenting a peptide.

The term “variable TCR α region” or “TCR α variable chain” or “variable domain” in the context of the invention refers to the variable region of a TCR α chain. The term “variable TCR β region” or “TCR β variable chain” in the context of the invention refers to the variable region of a TCR β chain.

The TCR loci and genes are named using the International Immunogenetics (IMGT) TCR nomenclature (IMGT Database, www.IMGT.org; Giudicelli, V., et al. IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

### Target

The term “epitope” in general refers to a site on an antigen, typically a (poly-) peptide, which a binding domain recognizes. The term “binding domain” in its broadest sense refers to an “antigen binding site”, i.e. characterizes a domain of a molecule which binds/interacts with a specific epitope on an antigenic target. An antigenic target may comprise a single epitope, but typically comprises at least two epitopes, and can include any number of epitopes depending on the size, conformation, and type of antigen. The term “epitope” in general encompasses linear epitopes and conformational epitopes. Linear epitopes are contiguous epitopes comprised in the amino acid primary sequence and typically include at least 2 amino acids or more. Conformational epitopes are formed by non-contiguous amino acids juxtaposed by folding of the target antigen, and in particular target (poly-) peptide.

Other embodiments refer to a cell comprising
(A) a NY-ESO1-specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40
   and
(B) a chimeric co-stimulatory receptor comprising
   an extracellular domain containing a polypeptide derived from PD-1
   a transmembrane domain, and
   an intracellular domain containing a polypeptide derived from 4-1BB,
   wherein the cell secretes more than two proteins.

### TCR Specific Sequence

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

The TCRs as described herein are specific for NY-ESO-1, in particular the NY-ESO-1 epitope SLLMWITQC (SEQ ID NO: 34) and exhibit only very low cross-reactivity to other epitopes or antigens.

Thus an exemplary TCR used in the combination of the invention comprises
a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40.

In some embodiments, the TCR comprises a variable TCR α region having an amino acid sequence which is at least 80% identical to SEQ ID NO: 41 and a variable TCR β region having an amino acid sequence which is at least 80% identical to SEQ ID NO: 42.

In specific embodiments the TCR comprises a variable TCR α region having the amino acid sequence of SEQ ID NO: 41 and a variable TCR β region having the amino acid sequence of SEQ ID NO: 42.

In sone embodiments, the TCR may comprise a TCR α chain having the amino acid sequence which is identical, or which is at least 80% identical to SEQ ID NO: 43, and a TCR β chain having the amino acid sequence which is identical or which is at least 80% identical to SEQ ID NO: 44.

In sone embodiments, the TCR may comprise a TCR α chain having the amino acid sequence of SEQ ID NO: 43, and a TCR β chain having the amino acid sequence of SEQ ID NO: 44.

### Modifications

In some embodiments, the amino acid sequence of the TCR and/or the chimeric co-stimulatory receptor may comprise one or more phenotypically silent substitutions.

“Phenotypically silent substitutions” are also named “conservative amino acid substitutions”. The concept of “conservative amino acid substitutions” is understood by the skilled artisan, and preferably means that codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. These variations can spontaneously occur, be introduced by random mutagenesis, or can be introduced by directed mutagenesis. Those changes can be made without destroying the essential characteristics of these polypeptides. The ordinarily skilled artisan can readily and routinely screen variant amino acids and/or the nucleic acids encoding them to determine if these variations substantially reduce or destroy the ligand binding capacity by methods known in the art.

The skilled person understands that also the nucleic acid encoding the TCR and/or the chimeric co-stimulatory receptor may be modified. Useful modifications in the overall nucleic acid sequence include codon optimization of the sequence. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

According to some embodiments of the invention the amino acid sequence of the TCR and/or the chimeric co-stimulatory receptor is modified to comprise a detectable label, a therapeutic agent or pharmacokinetic modifying moiety.

Non-limiting examples for detectable labels are radiolabels, fluorescent labels, nucleic acid probes, enzymes and contrast reagents. Therapeutic agents which may be associated with the TCRs include radioactive compounds, immune-modulators, enzymes or chemotherapeutic agents. The therapeutic agents could be enclosed by a liposome linked to TCR so that the compound can be released slowly at the target site. This will avoid damage during the transport in the body and ensure that the therapeutic agent, e.g. toxin, has maximum effect after binding of the TCR to the relevant antigen presenting cells. Other examples for therapeutic agents are:
peptide cytotoxins, i.e. proteins or peptides with the ability to kill mammalian cells, such as ricin, diphtheria toxin, Pseudomonas bacterial exotoxin A, DNase and RNase. Small molecule cytotoxic agents, i.e. compounds with the ability to kill mammalian cells having a molecular weight of less than 700 Daltons. Such compounds could contain toxic metals capable of having a cytotoxic effect. Furthermore, it is to be understood that these small molecule cytotoxic agents also include pro-drugs, i.e. compounds that decay or are converted under physiological conditions to release cytotoxic agents. Such agents may for example include docetaxel, gemcitabine, cisplatin, maytansine derivatives, rachelmycin, calicheamicin, etoposide, ifosfamide, irinotecan, porfimer sodium photofrin II, temozolomide, topotecan, trimetrexate glucoronate, mitoxantrone, auristatin E, vincristine and doxorubicin; radionuclides, such as, iodine 131, rhenium 186, indium 111, yttrium 90, bismuth 210 and 213, actinium 225 and astatine 213. The association of the radionuclides with the TCRs or derivatives thereof may for example be carried out by chelating agents; immune-stimulators, also known as immunostimulants, i.e. immune effector molecules which stimulate immune response. Exemplary immune-stimulators are cytokines such as IL-2 and IFN-γ, antibodies or fragments thereof, including anti-T cell or NK cell determinant antibodies (e.g anti-CD3, anti-CD28 or anti-CD16); alternative protein scaffolds with antibody like binding characteristics; Superantigens, i.e. antigens that cause non-specific activation of T cells resulting in polyclonal T cell activation and massive cytokine release, and mutants thereof, chemokines such as IL-8, platelet factor 4, melanoma growth stimulatory protein, etc. complement activators; xenogeneic protein domains, allogeneic protein domains, viral/bacterial protein domains, viral/bacterial peptides.

The therapeutic agent may preferably be selected from the group consisting of an immune effector molecule, a cytotoxic agent and a radionuclide. Preferably, the immune effector molecule is a cytokine.

The pharmacokinetic modifying moiety may be for example at least one polyethylene glycol repeating unit, at least one glycol group, at least one sialyl group or a combination thereof. The association of at least one polyethylene glycol repeating unit, at least one glycol group, at least one sialyl group may be caused in a number of ways known to those skilled in the art. In a preferred embodiment the units are covalently linked to the TCR. The TCRs according to the invention can be modified by one or several pharmacokinetic modifying moieties. In particular, the soluble form of the TCR is modified by one or several pharmacokinetic modifying moieties. The pharmacokinetic modifying moiety may achieve beneficial changes to the pharmacokinetic profile of the therapeutic, for example improved plasma half-life, reduced or enhanced immunogenicity, and improved solubility.

The TCR and/or the chimeric co-stimulatory receptor can be modified by attaching additional functional moieties, e.g. for reducing immunogenicity, increasing hydrodynamic size (size in solution) solubility and/or stability (e.g. by enhanced protection to proteolytic degradation) and/or extending serum half-life.

Other useful functional moieties and modifications include “suicide” or “safety switches” that can be used to shut off effector host cells carrying an inventive TCR in a patient's body. An example is the inducible Caspase 9 (iCasp9) “safety switch” described by Gargett and Brown Front Pharmacol. 2014; 5: 235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” “safety switches” are known in the art, e.g. Herpes Simplex Virus thymidine kinase (HSV-TK), expression of CD20 and subsequent depletion using anti-CD20 antibody or myc tags (Kieback et al, Proc Natl Acad Sci USA. 2008 Jan. 15; 105(2):623-8).

TCRs with an altered glycosylation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. Similarly, deglycosylation (i.e., removal of carbohydrate moieties present on the binding molecule) may be accomplished chemically, e.g. by exposing the TCRs to trifluoromethanesulfonic acid, or enzymatically by employing endo- and exo-glycosidases.

It is also conceivable to add a drug such as a small molecule compound to the TCR, in particular a soluble form of the inventive TCR. Linkage can be achieved via covalent bonds, or non-covalent interactions such as through electrostatic forces. Various linkers, known in the art, can be employed in order to form the drug conjugates.

The TCR, in particular a soluble form of the inventive TCR can additionally be modified to introduce additional domains which aid in identification, tracking, purification and/or isolation of the respective molecule (tags). Thus, in some embodiments, the TCR α chain or the TCR β chain may be modified to comprise an epitope tag.

Epitope tags are useful examples of tags that can be incorporated into the TCR of the invention. Epitope tags are short stretches of amino acids that allow for binding of a specific antibody and therefore enable identification and tracking of the binding and movement of soluble TCRs or host cells within the patient's body or cultivated (host) cells. Detection of the epitope tag, and hence, the tagged TCR, can be achieved using a number of different techniques.

Tags can further be employed for stimulation and expansion of host cells carrying an inventive TCR by cultivating the cells in the presence of binding molecules (antibodies) specific for said tag.

In general, the TCR can be modified in some instances with various mutations that modify the affinity and the off-rate of the TCR with the target antigen. In particular, the mutations may increase the affinity and/or reduce the off-rate. Thus, the TCR may be mutated in at least one CDR and the variable domain framework region thereof.

However, in a preferred embodiment the CDRs of the TCR are not modified or in vitro affinity maturated such as for the TCRs in the examples. This means that the CDRs have naturally occurring sequences. This can be advantageous, since in vitro affinity maturation may lead to immunogenicity to the TCR molecule. This may lead to the production of anti-drug antibodies decreasing or inactivating the therapeutic effect and the treatment and/or induce adverse effects.

The mutation may be one or more substitution(s), deletion(s) or insertions(s). These mutations may be introduced by any suitable method known in the art, such as polymerase chain reaction, restriction enzyme-based cloning, ligation independent cloning procedures, which are described for Example in Sambrook, Molecular Cloning—4^{th} Edition (2012) Cold Spring Harbor Laboratory Press.

Theoretically, unpredictable TCR specificity with the risk for cross-reactivity can occur due to mispairing between endogenous and exogenous TCR chains. To avoid mispairing of TCR sequences, the recombinant TCR sequence may be modified to contain murinized or minimal murinized Cα and Cβ regions, a technology that has been shown to efficiently enhance correct pairing of several different transduced TCR chains. Murinization of TCRs (i.e. exchanging the human Cα and Cβ regions by their murine counterparts) is a technique that is commonly applied in order to improve cell surface expression of TCRs in host cells. Without wishing to be bound by specific theory, it is thought that murinized TCRs associate more effectively with CD3 co-receptors; and/or that preferentially pair with each other and are less prone to form mixed TCRs on human T cells genetically modified ex vivo to express the TCRs of desired antigenic specificity, but still retaining and expressing their “original” TCRs.

Nine amino acids responsible for the improved expression of murinized TCRs have been identified (Sommermeyer and Uckert, J Immunol. 2010 Jun. 1; 184(11):6223-31) and it is envisaged to substitute one or all of the amino acid residues in the TCRs α and/or β chain constant region for their murine counterpart residues. This technique is also referred to as “minimal murinization”, and offers the advantage of enhancing cell surface expression while, at the same time, reducing the number of “foreign” amino acid residues in the amino acid sequence and, thereby, the risk of immunogenicity.

Some embodiments refer to an isolated TCR as described herein, wherein the TCR is of the single chain type, wherein the TCR α chain and the TCR β chain are linked by a linker sequence.

A suitable single chain TCR form comprises a first segment constituted by an amino acid sequence corresponding to a variable TCR α region, a second segment constituted by an amino acid sequence corresponding to a variable TCR β region fused to the N terminus of an amino acid sequence corresponding to a TCR β chain constant region extracellular sequence, and a linker sequence linking the C terminus of the first segment to the N terminus of the second segment. Alternatively, the first segment may be constituted by an amino acid sequence corresponding to a TCR β chain variable region, the second segment may be constituted by an amino acid sequence corresponding to a TCR α chain variable region sequence fused to the N terminus of an amino acid sequence corresponding to a TCR α chain constant region extracellular sequence. The above single chain TCRs may further comprise a disulfide bond between the first and second chains, and wherein the length of the linker sequence and the position of the disulfide bond being such that the variable domain sequences of the first and second segments are mutually orientated substantially as in native T cell receptors. More specifically the first segment may be constituted by an amino acid sequence corresponding to a TCR α chain variable region sequence fused to the N terminus of an amino acid sequence corresponding to a TCR α chain constant region extracellular sequence, the second segment may be constituted by an amino acid sequence corresponding to a TCR β chain variable region fused to the N terminus of an amino acid sequence corresponding to TCR β chain constant region extracellular sequence, and a disulfide bond may be provided between the first and second chains. The linker sequence may be any sequence which does not impair the function of the TCR.

In the context of the present invention, a “functional” TCR α and/or β chain fusion protein shall mean a TCR or TCR variant, for example modified by addition, deletion or substitution of amino acids, that maintains at least substantial biological activity. In the case of the α and/or β chain of a TCR, this shall mean that both chains remain able to form a TCR (either with a non-modified α and/or β chain or with another inventive fusion protein α and/or β chain) which exerts its biological function, in particular binding to the specific peptide-MHC complex of said TCR, and/or functional signal transduction upon specific peptide:MHC interaction.

In specific embodiments the TCR may be modified, to be a functional TCR α and/or β chain fusion protein, wherein said epitope-tag has a length of between 6 to 15 amino acids, preferably 9 to 11 amino acids. In another embodiment the TCR may be modified to be a functional T-cell receptor (TCR) α and/or β chain fusion protein wherein said TCR α and/or β chain fusion protein comprises two or more epitope-tags, either spaced apart or directly in tandem. Embodiments of the fusion protein can contain 2, 3, 4, 5 or even more epitope-tags, as long as the fusion protein maintains its biological activity/activities (“functional”).

Preferred is a functional TCR α and/or β chain fusion protein according to the present invention, wherein said epitope-tag is selected from, but not limited to, CD20 or Her2/neu tags, or other conventional tags such as a myc-tag, FLAG-tag, T7-tag, HA (hemagglutinin)-tag, His-tag, S-tag, GST-tag, or GFP-tag. myc, T7, GST, GFP tags are epitopes derived from existing molecules. In contrast, FLAG is a synthetic epitope tag designed for high antigenicity (see, e.g., U.S. Pat. Nos. 4,703,004 and 4,851,341). The myc tag can preferably be used because high quality reagents are available to be used for its detection. Epitope tags can of course have one or more additional functions, beyond recognition by an antibody. The sequences of these tags are described in the literature and well known to the person of skill in art.

### Chimeric Co-Stimulatory Receptor

The chimeric co-stimulatory receptor used in combination with the antigen specific TCR (the NY-ESO specific TCR) comprises
an extracellular domain containing a polypeptide derived from PD-1,
a transmembrane domain, and
an intracellular domain containing a polypeptide derived from 4-1BB.

The chimeric co-stimulatory receptor used in combination with the antigen-specific TCR, such as the NY-ESO-1 specific TCR, herein may particularly comprise an extracellular domain containing the extracellular domain derived from PD-1 (e.g. human PD-1). In this context, the term “derived from” particularly means that the polypeptide contained in the extracellular domain comprises at least a part of PD-1 (e.g. human PD-1), preferably the extracellular domain of PD-1, respectively. The chimeric co-stimulatory receptor comprising an extracellular domain derived from PD-1 has binding activity for PD-L1, PD-L2 or other inhibitory ligands of PD-1. As used herein, the term “derived from” PD-1 also allows that up to 0, 1, 2, 3, 4, 5, 6, 7, 8, 9, 10 or more amino acids are substituted, deleted, and/or inserted compared to a native sequence of PD-1 (e.g. human PD-1) or part thereof (e.g., extracellular domain).

In one embodiment the extracellular domain containing a polypeptide derived from PD-1 comprises sequence set out in SEQ ID NO: 28 or amino acid sequence which is at least 80% identical to SEQ ID NO: 28. In a specific embodiment, the extracellular domain containing a polypeptide derived from PD-1 comprises sequence set out in SEQ ID NO: 28.

In one embodiment of the present invention, the chimeric co-stimulatory receptor comprises an extracellular domain containing a polypeptide derived from PD-1 comprises an amino acid sequence with up to 0, 1, 2, 3, 4, 5, 6, 7, 8, 9, or 10 amino acid substitutions (preferably conservative or highly conservative substitutions), deletions and/or insertions compared to the amino acid sequence of the extracellular domain of human or murine PD-1, e.g., of human PD-1 as depicted in SEQ ID NO: 28.

The chimeric co-stimulatory receptor used in combination with the antigen-specific TCR, such as the NY-ESO-1 specific TCR, herein further comprise a transmembrane domain operably linked between the extracellular domain and the intracellular domain. Generally, the transmembrane domain is not limited to a specific transmembrane domain. Preferably, the transmembrane domain allows stable anchorage of the fusion protein in the membrane of a cell expressing the fusion protein (e.g., a T cell) and further allows binding of the extracellular domain to PD-L1, respectively, and, upon binding to PD-L1, allows signaling transduction to the intracellular domain containing a polypeptide derived from 4-1BB.

In a preferred embodiment, the transmembrane domain of the chimeric co-stimulatory receptor is derived from PD-1. In one embodiment the transmembrane domain comprises a sequence set out in SEQ ID NO: 30 or amino acid sequence, which is at least 80% identical to SEQ ID NO: 30. In a specific embodiment, the transmembrane domain containing a polypeptide derived from PD-1 comprises sequence set out in SEQ ID NO: 30.

The chimeric co-stimulatory receptors used in combination with the antigen-specific TCR, such as the NY-ESO-1 specific TCR, herein may particularly comprise an intracellular domain containing a polypeptide derived from 4-1BB (also termed “41BB”), preferably the intracellular domain of 4-1BB (e.g human 4-1BB). In this context, the term “derived from” particularly means that the polypeptide contained in the intracellular domain comprises at least a part of 4-1BB (e.g. human 4-1BB), preferably the intracellular domain of 4-1BB, respectively. The chimeric co-stimulatory receptor comprising an intracellular domain derived from 4-1BB is capable of increasing the proliferation rate of a T cell expressing said chimeric co-stimulatory receptor upon stimulation with PD-L1, PD-L2 or another inhibitory ligand of PD-1 and/or is capable of increasing the effector function (such as increased IFN-γ release and/or increased cytotoxicity) of a T cell expressing said chimeric co-stimulatory receptor compared to a corresponding T cell not expressing the chimeric co-stimulatory receptor. As used herein, the term “derived from” 4-1BB also allows that up to 0, 1, 2, 3, 4, 5, 6, 7, 8, 9, 10 or more amino acids are substituted, deleted, and/or inserted compared to a native sequence of 4-1BB (human or murine, preferably human 4-1BB) or part thereof (e.g., intracellular domain). In one embodiment the intracellular domain containing a polypeptide derived from 4-1BB comprises sequence set out in SEQ ID NO: 32 or amino acid sequence which is at least 80% identical to SEQ ID NO: 32. In a specific embodiment, the intracellular domain containing a polypeptide derived from 4-1BB comprises sequence set out in SEQ ID NO: 32.

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

### Nucleic Acids, Nucleic Acid Compositions and Vectors

The invention encompasses nucleic acids encoding antigen-specific TCRs, in particular the NY-ESO-1 specific TCRs as described herein, as well as the corresponding nucleic acid compositions and vectors comprising said nucleic acids.

The nucleotide sequences encoding the relevant regions and domains of NY-ESO-specific TCR are set out in Table 1:

| Peptide seq. | |
|---|---|
| SEQ ID NO | Description |
| 35 | TCR1 α chain CDR1 |
| 36 | TCR1 α chain CDR2 |
| 37 | TCR1 α chain CDR3 |
| 38 | TCR1 β chain CDR1 |
| 39 | TCR1 β chain CDR2 |
| 40 | TCR1 β chain CDR3 |
| 41 | TCR1 α chain variable region |
| 42 | TCR1 β chain variable region |
| 43 | TCR β chain |
| 42 | TCR α chain |

The nucleotide sequences encoding the relevant regions and domains of the chimeric co-stimulatory receptor are set out in Table 2:

TABLE 2

| Peptide seq. | nucleotide seq. | |
|---|---|---|
| SEQ ID NO | SEQ ID NO | Description |
| 26 | 27 | chimeric co-stimulatory receptor |
| | | comprising a PD-1 extracellular domain, |
| | | PD-1 transmembrane domain and a 4-1BB |
| | | intracellular domain |
| 28 | 29 | PD-1 extracellular domain |
| 30 | 31 | PD-1 transmembrane domain |
| 32 | 33 | 4-1BB intracellular domain |

Moreover, a further aspect relates to a composition comprising
a nucleic acid encoding a NY-ESO-1 specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, one aspect relates to a nucleic acid comprising
a nucleic acid encoding a NY-ESO-1 specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

A further aspect refers to a vector comprising the nucleic acid comprising the sequences for the NY-ESO-1-specific TCR and the chimeric co-stimulatory receptor. Also cells comprising the nucleic acid composition and/or the vector are encompassed.

“Nucleic acid molecule” generally means a polymer of DNA or RNA, which can be single-stranded or double-stranded, synthesized or obtained (e.g., isolated and/or purified) from natural sources which can contain natural, non-natural or altered nucleotides, and which can contain a natural, non-natural or altered internucleotide linkage, such as a phosphoroamidate linkage or a phosphorothioate linkage instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from Genscript, Thermo Fisher and similar companies). See, for example, Sambrook et al., a nucleic acid can be chemically synthesized using naturally occurring nucleotides or variously modified nucleotides (see for example Sambrook et al. 2001) designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs and/or chimeric co-stimulatory receptors, polypeptides, or proteins, or functional portions or functional variants thereof.

For example, the present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the TCR described herein. Such variant nucleotide sequence encodes a functional TCR that specifically recognizes NY-ESO-1, especially NY-ESO-1 epitope of SEQ ID NO: 34.

For example, the present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to chimeric co-stimulatory receptor described herein. Such variant nucleotide sequence encodes a functional chimeric co-stimulatory receptor as described herein.

As already described elsewhere herein, the nucleic acid encoding the TCR and/or chimeric co-stimulatory receptor may be modified. Useful modifications in the overall nucleic acid sequence may be codon optimization. Alterations may be made which lead to conservative substitutions within the translated amino acid sequence. With regard to TCRs, these variations can be made in complementarity determining and non-complementarity determining regions of the amino acid sequence of the TCR chain that do not affect function. Usually, additions and deletions should not be performed in the CDR3 region.

Another embodiment refers to a vector comprising the nucleic acid encoding the TCR and the chimeric co-stimulatory receptor as described herein.

The vector is preferably a plasmid, shuttle vector, phagemide, cosmid, expression vector, retroviral vector, adenoviral vector or particle and/or vector to be used in gene therapy.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence (e.g. a nucleic acid of the invention). The vector may comprise DNA or RNA and/or comprise liposomes and/viral particles The vector may be a plasmid, shuttle vector, phagemide, cosmid, expression vector, retroviral vector, lentiviral vector, adenoviral vector or particle and/or vector to be used in gene therapy. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

Preferably, the vector is an expression vector. More preferably, the vector is a retroviral, more specifically a γ-retroviral or lentiviral vector.

The skilled person understands that the chimeric co-stimulatory receptor sequence and the TCR chain TCR-α and TCR-β chain sequences can be included in one nucleic acid, e.g. one vector. In this case the sequences are linked with either an internal ribosomal entry site (IRES) sequence or the 2A peptide sequence derived from a porcine teschovirus (P2A) or derived from other species like Thosea asigna virus 2A peptide (T2A) or foot and mouth disease virus 2A peptide (F2A) (as described in Szymczak et al.: Development of 2A peptide-based strategies in the design of multicistronic vectors) resulting in the expression a single messenger RNA (mRNA) molecule under the control of the viral promoter within the transduced cell.

In specific embodiments, the cell may comprise the nucleic acid encoding the TCR and the chimeric co-stimulatory receptor as described herein or the vector comprising said nucleic acid.

The term “transfection” and “transduction” are interchangeable and refer to the process by which an exogenous nucleic acid sequence is introduced in a host cell, e.g. in a eukaryotic host cell. It is noted that introduction or transfer of nucleic acid sequences is not limited to the mentioned methods but can be achieved by any number of means including electroporation, microinjection, gene gun delivery, lipofection, superfection and the mentioned infection by retroviruses or other suitable viruses for transduction or transfection. The method of cloning and exogenous expression of the TCR is for example described in Engels et al. (Relapse or eradication of cancer is predicted by peptide-major histocompatibility complex affinity. Cancer Cell, 23(4), 516-26. 2013). The transduction of primary human T cells with a lentiviral vector is, for example, described in Cribbs “simplified production and concentration of lentiviral vectors to achieve high transduction in primary human T cells” BMC Biotechnol. 2013; 13: 98.

The cell described and provided in context with the present invention comprising the nucleic acid molecule or the vector as described and provided herein is preferably able to stably or transiently (e.g., stably) express (either constitutively or conditionally) the antigen specific TCR, such as the NY-ESO-1 specific TCR, and the chimeric co-stimulatory receptor of the present invention. The host cell may generally be transduced or transformed by any method with any suitable nucleic acid molecule or vector. In one embodiment, the host cell is transduced with a retroviral or lentiviral (e.g., retroviral) vector comprising a nucleic acid molecule encoding the fusion protein of the present invention or parts thereof (e.g., ECD, TMD, and/or ICD) as described above.

In some embodiments, the cell (including e.g. the target specific immune cell or the cell of the population of cells as referred herein) is a peripheral blood lymphocyte (PBL) or a peripheral blood mononuclear cell (PBMC). The cell may be a natural killer cell or a T cell. Preferably, the cell is a T cell. The T cell may be a CD4+ or a CD8+ T cell. In some embodiments the cell is a stem cell like memory T cell.

Stem cell-like memory T cells (TSCM) are a less-differentiated subpopulation of CD8+ or CD4+ T cells, which are characterized by the capacity of self-renewal and to persist long-term. Once these cells encounter their antigen in vivo, they differentiate further into central memory T cells (TCM), effector memory T cells (TEM) and terminally differentiated effector memory T cells (TEMRA) with some TSCM remaining quiescent (Flynn et al., Clinical & Translational Immunology (2014). These remaining TSCM cells show the capacity to build a durable immunological memory in vivo and therefore are considered an important T cell subpopulation for adoptive T cell therapy (Lugli et al., Nature Protocols 8, 33-42 (2013) Gattinoni et al., Nat. Med. 2011 October; 17(10): 1290-1297). Immune-magnetic selection can be used in order to restrict the T cell pool to the stem cell memory T cell subtype see (Riddell et al. 2014, Cancer Journal 20(2): 141-44).

### Pharmaceutical Compositions, Medical Treatments and Kits

Another aspect of the invention refers to pharmaceutical composition comprising the cell comprising the antigen-specific TCR, such as the NY-ESO-1 specific TCR, and the chimeric co-stimulatory receptor or comprising nucleic acid molecules encoding said molecules as described herein, the nucleic acid encoding the antigen-specific TCR, such as the NY-ESO-1 specific TCR, and the chimeric co-stimulatory receptor, a composition comprising nucleic acids encoding the antigen-specific TCR, such as the NY-ESO-1 specific TCR, and nucleic acids encoding the chimeric co-stimulatory receptor, the corresponding vector as described herein.

Those active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” defines a non-toxic material, which does not interfere with effectiveness of the biological activity of the active component. The choice of the carrier is dependent on the application.

The pharmaceutical composition may contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in “Remington's Pharmaceutical Sciences”, Mack Publishing Co., Easton, PA, latest edition. An appropriate application is a parenteral application, for example intramuscular, subcutaneous, intramedular injections as well as intrathecal, direct intraventricular, intravenous, intranodal, intraperitoneal or intratumoral injections. The intravenous injection is the preferred treatment of a patient.

According to a preferred embodiment, the pharmaceutical composition is an infusion or an injection.

An injectable composition is a pharmaceutically acceptable fluid composition comprising at least one active ingredient, e.g. an expanded T cell population (for example autologous or allogenic to the patient to be treated) comprising the antigen-specific TCR, such as the NY-ESO-1 specific TCR, and the chimeric co-stimulatory receptor. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this disclosure are conventional; appropriate formulations are well known to those of ordinary skill in the art.

Typically, the pharmaceutical composition comprises at least one pharmaceutically acceptable carrier.

Accordingly, another aspect of the invention refers to the cell as described herein, the composition as described herein, the nucleic acid as described herein, and/or the vector as described herein for use as a medicament.

Some embodiments refer to the to the cell as described herein, the composition as described herein, the nucleic acid as described herein, and/or the vector for use in the treatment of cancer.

Accordingly, also a method for treating cancer in a human or non-human animal in need thereof, comprising administering to said human or non-human animal the cell as described herein, the composition as described herein, the nucleic acid as described herein, and/or the vector as describe herein is considered.

In one embodiment the cancer is a hematological cancer or a solid tumor.

Hematological cancers also called blood cancers which do not form solid tumors and therefore are dispersed in the body. Examples of hematological cancers are leukemia, lymphoma or multiple myeloma. There are two major types of solid tumors, sarcomas and carcinomas. Sarcomas are for example tumors of the blood vessel, bone, fat tissue, ligament, lymph vessel, muscle or tendon. In a specific embodiment the cancer is a solid tumor.

In one embodiment, the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, carcinoma, sarcoma or osteosarcoma.

Compositions comprising the modified T cells as described herein can be utilized in methods and compositions for adoptive immunotherapy in accordance with known techniques, or variations thereof that will be apparent to those skilled in the art based on the instant disclosure.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The number of cells for an effective treatment in the composition is typically greater than 10 cells, and up to 10⁶, up to and including 10⁸ or 10⁹ cells and can be more than 10¹⁰ cells. The number of cells will depend upon the ultimate use for which the composition is intended as will the type of cells included therein. For uses provided herein, the cells are generally in a volume of a liter or less, can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells. Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body. In a composition intended to be administered by injection, one or more of a surfactant, preservative, wetting agent, dispersing agent, suspending agent, buffer, stabilizer and isotonic agent may be included.

The viral vector particles comprising a nucleotide sequence encoding the antigen-specific TCR, such as the NY-ESO-1 specific TCR, and the chimeric co-stimulatory receptor provided herein, can be packaged as kits. Kits can optionally include one or more components such as instructions for use, devices, and additional reagents, and components, such as tubes, containers and syringes for practice of the methods. Exemplary kits can include the nucleic acids encoding the recombinant TCRs and the chimeric co-stimulatory receptors, the recombinant polypeptides, or viruses provided herein, and can optionally include instructions for use, a device for detecting a virus in a subject, a device for administering the compositions to a subject, and a device for administering the compositions to a subject.

Kits comprising polynucleotides encoding the antigen-specific TCR, such as the NY-ESO-1 specific TCR, and the chimeric co-stimulatory receptor are also contemplated herein. Kits comprising a viral vector encoding a sequence of interest (e.g., a recombinant TCR) and optionally, a polynucleotide sequence encoding an immune checkpoint inhibitor are also contemplated herein.

Kits contemplated herein also include kits for carrying out the methods for detecting the presence of polynucleotides encoding any one or more of the TCRs and/or the chimeric co-stimulatory receptors disclosed herein. In particular, such diagnostic kits may include sets of appropriate amplification and detection primers and other associated reagents for performing deep sequencing to detect the polynucleotides encoding TCRs and/or the chimeric co-stimulatory receptors disclosed herein. In further embodiments, the kits herein may comprise reagents for detecting the TCRs and/or the chimeric co-stimulatory receptors disclosed herein, such as antibodies or other binding molecules. Diagnostic kits may also contain instructions for determining the presence of the polynucleotides encoding the TCRs and/or the chimeric co-stimulatory receptors disclosed herein or for determining the presence of the TCRs and/or the chimeric co-stimulatory receptors disclosed herein. A kit may also contain instructions. Instructions typically include a tangible expression describing the components included in the kit, and methods for administration, including methods for determining the proper state of the subject, the proper dosage amount, and the proper administration method. Instructions can also include guidance for monitoring the subject over the duration of the treatment time.

Kits provided herein also can include a device for administering a composition described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus of the kit will be compatible with the virus of the kit; for example, a needle-less injection device such as a high pressure injection device can be included in kits with viruses not damaged by high pressure injection, but is typically not included in kits with viruses damaged by high pressure injection.

### Cytokine Release

The cells expressing both the TCR and the costimulatory molecule, such as PD1-4BB, show enhanced cytokine release. Cytokines/lytic protein that are secreted in high amounts by the population are inter alia IFN-γ, Gzm-B, and IP-10, MIP-1β.

Surprisingly, immune cells, in particular TCR-T cells expressing chimeric-costimulatory receptor as defined herein show a higher polyfunctionality compared to TCR-T cells lacking chimeric-costimulatory receptor as defined herein.

Higher polyfunctionality of transgenic T cells point to a higher functionality and anti-tumor activity in vivo and correlates with clinical outcome.

The term “polyfunctional” means that the cell secretes at least 2, such as at least three, at least four, at least five proteins.

The skilled person understands that the cell secretes the proteins after antigen specific stimulation, for example when the cell is contacted with an antigen presenting cell presenting the antigen in a MHC molecule.

In particular, the cell population comprises a significant percentage of cells, such as 0.1%, 0.2%, 0.3%, 0.4%, 0.5%, 1%, 1.5%, 2%, 3%, 4%, 4.5% polyfunctional cells.

In particular, the cell population comprises a significant percentage of cells expressing at least 1%, preferably at least 1.5%, more preferably 1.75% 3 or more proteins.

In some embodiments the polyfunctional strength index is 80 or more, preferably 90 or more, more preferably 100 or more.

The poly-functional strength index (PSI) is calculated by multiplying the intensities of the various secreted cytokines and the percentage of polyfunctional T cells.

In particular we could show that a cell population expressing both the target specific, e.g. NY-ESO-1 specific, TCR and the chimeric-costimulatory receptor as defined herein comprises cells which secrete at least two proteins, e.g. at least three proteins, at least four proteins, at least 5 proteins.

Hence, another aspect of the invention refers to an in vitro assay indicating the potency of a population of T cells by indicating whether a population of T cells comprises a significant proportion of polyfunctional T cells as defined herein.

Thus, in general, desired are highly potent target specific T cells, that secrete at least two, at least three, at least four, at least five proteins selected from the group of effector proteins, stimulatory cytokines and chemo-attractive cytokines.

In particular, the invention refers to an in vitro assay indicating the potency of a population of immune cells, comprising the steps
measuring the proportion of polyfunctional immune cells of a first population of cells,
assessing the potency of the first population of immune cells and assessing the potency of a second population of T cells, wherein an increased proportion of polyfunctional cells in the first population compared the second population indicates that the first population has a higher potency than the second population.

The measurement of the proportion of polyfunctional immune cells may be carried out by the IsoLight® technology (Isoplexis).

Accordingly, T cell populations comprising target specific T cells wherein the cell population comprises T cells that secrete at least two, at least three, at least four, at least five proteins selected from the group of effector proteins, stimulatory cytokines and chemo-attractive cytokines are desired.

The effector proteins may be selected from the group consisting of Gzm-B, IFN-γ, Perforin, TNF-α, TNF-β, MIP-1α.

In one embodiment, the stimulatory cytokines may be selected from the group consisting of GM-CSF, IL-2, IL-7, IL-8, IL-9, IL-12. In one embodiment, the stimulatory protein is selected from GM-CSF, IL-2 and IL-8. In one embodiment the stimulatory protein protein is GM-CSF.

The chemo-attractive cytokines may be selected from IP-10 and MIP-1β.

In some embodiments, each of the cells secreting at least two proteins, secretes at least one of the proteins selected from the group consisting of Gzm-B, IFN-γ, Perforin, TNF-α, TNF-β, MIP-1α, GM-CSF, IL-2, IL-5, IL-7, IL-8, IL-9, IL-12, IP-10 and MIP-10. In some embodiments, each of the cells secreting at least two proteins, secretes at least one of the proteins selected from the group consisting of Gzm-B, IFN-γ, Perforin, TNF-α, TNF-β, GM-CSF, IL-2, IL-8, MIP-1β and IP-10. In some embodiments, each of the cells secreting at least two proteins, secretes at least one of the proteins selected from the group consisting of IFN-γ, Gzm-B, GM-CSF, MIP-1β and IP-10. In some embodiments, each of the cells secreting at least two proteins, secretes at least one of the proteins selected from the group consisting of IFN-γ, Gzm-B, GM-CSF. In some embodiments, each of the cells secreting at least two proteins, secretes at least one of the proteins selected from the group consisting of IFN-γ, Gzm-B, and IP-10. In some embodiments, each of the cells secreting at least two proteins, secretes at least one of the proteins selected from the group consisting of IFN-γ and Gzm-B.

Typically, the cell population does not comprise cells which secrete significant amounts of IL-6 and/or IL-10.

Other embodiments refer to T cells expressing (A) a NY-ESO-1 specific TCR as defined herein and (B) a chimeric co-stimulatory receptor as defined herein, wherein the T cells secrete at least two, at least three, at least four, at least five proteins selected from the group consisting of effector proteins, stimulatory cytokines and chemo-attractive cytokines.

Accordingly, some embodiments refer to cell population comprising T cells expressing (A) a NY-ESO-1 specific TCR as defined herein and (B) a chimeric co-stimulatory receptor as defined herein, wherein the cell population comprises T cells secreting at least two, at least three, at least four, at least five proteins selected from the group consisting of effector proteins, stimulatory cytokines and chemo-attractive cytokines.

### EXPERIMENTS

### Example 1: TCR-T Cells Targeting NY-ESO-1 Expressing PD1-41BB Show a Higher Polyfunctionality Compared to TCR-T Cells Targeting NY-ESO-1 Lacking PD1-41BB

TCR-transgenic T cells with or without PD1-41BB were analyzed regarding their single-cell polyfunctionality (release of 2 or more proteins) using the IsoLight® technology (IsoPlexis). So-called “polyfunctional” T cells secrete multiple proteins (in particular cytokines and other effector proteins such as Gzm-B), thereby enabling a whole variety of effector functions and a highly effective immune response. After co-culture with the PD-L1-overexpressing, NY-ESO-1-positive MelA375 or Mel624.38 tumor cell line for 20 h, single CD8⁺ T cells were separated from tumor cells using CD8 Microbeads (Miltenyi Biotec) and loaded onto IsoCode chips (IsoPlexis). The secretion of 32 T cell cytokines/proteins was analyzed using the IsoLight device and the IsoSpeak software (IsoPlexis). TCR-T cells expressing PD1-41BB showed a higher percentage of poly-functional T cells, meaning T cell secreting 2 or more proteins, compared to TCR-T cells lacking PD1-41BB (FIG. 1 A). The poly-functional strength index (PSI) was calculated by multiplying the intensities of the various secreted proteins/cytokines and the percentage of polyfunctional T cells. TCR-T cells expressing PD1-41BB showed a higher PSI compared to TCR-T cells lacking PD1-41BB (FIG. 1 B).

Classification of the various proteins/cytokines released revealed a high contribution of effector proteins (Gzm-B, IFN-γ, MIP-1a, Perforin, TNF-α, TNF-β) and stimulatory cytokines (GM-CSF, IL-12, IL-2, IL-5, IL-8, IL-9) to the superior PSI, followed by chemo-attractive cytokines (IP-10, MIP-1β, RANTES). Regulatory (IL-4, IL-10, IL-22, sCD137, TGF-β1) and inflammatory (CP-1, IL-17F, IL8) cytokines were released to a lower extent. Detailed analysis of the single-cell poly-protein/cytokine release using a polyfunctionality heat map showed that TCR-T cells with and without PD1-41BB differed in their poly-protein/cytokine signature (FIG. 1 C). Notably, TCR-T cells expressing PD1-41BB contained a higher proportion of single cells secreting 2-6 proteins/cytokines simultaneously, pointing to a superior functionality compared to TCR-T cells lacking PD1-41BB. The main proteins/cytokines released by TCR-T cells expressing PD1-41BB were Gzm-B, IFN-γ, GM-CSF and MIP-1β, belonging to the families of effector proteins and stimulatory cytokines and thus pointing to a high TCR-T cell functionality and potential anti-tumor activity.

### Example 2: Improved Effector Function of NY-ESO-1 TCR-T Cells Co-Expressing Chimeric PD1-41BB Co-Stimulatory Receptor

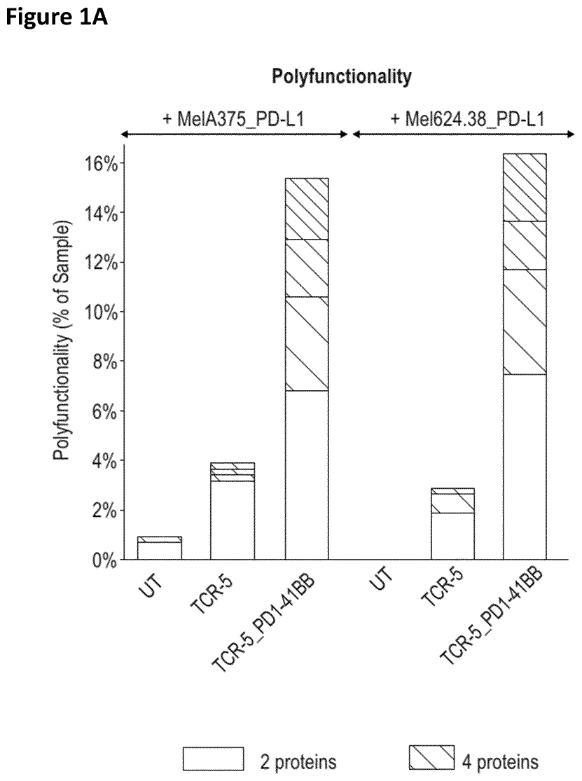

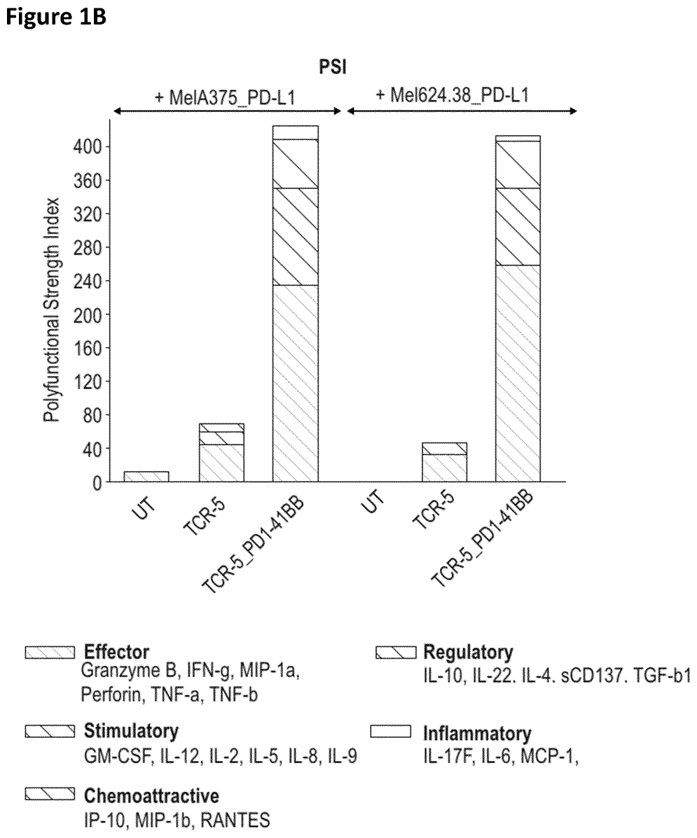

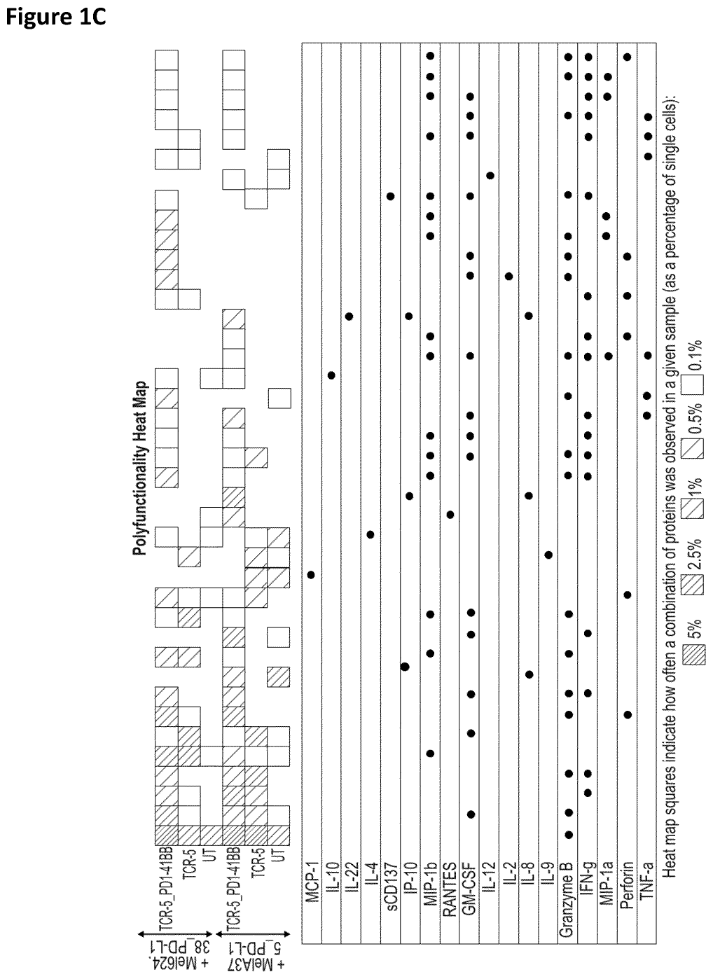

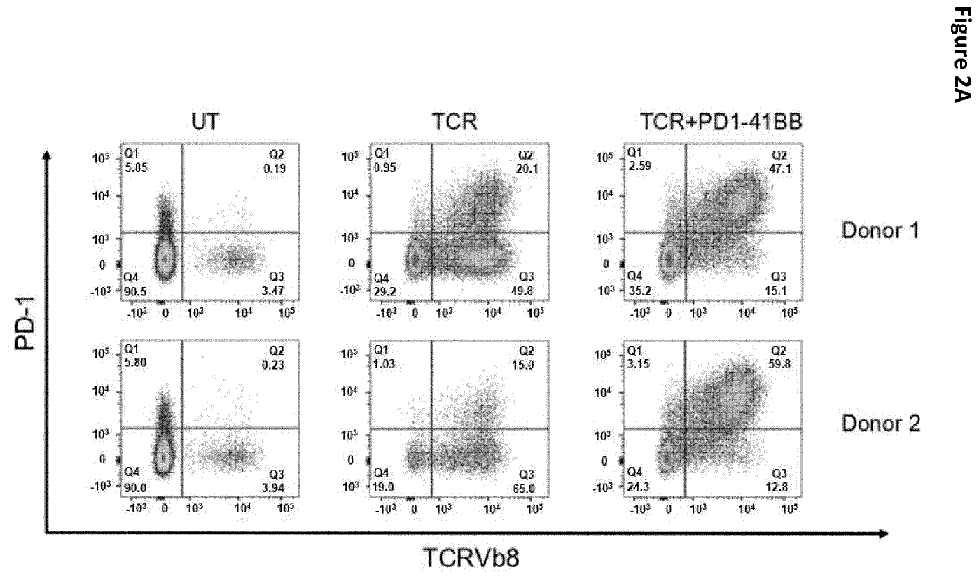

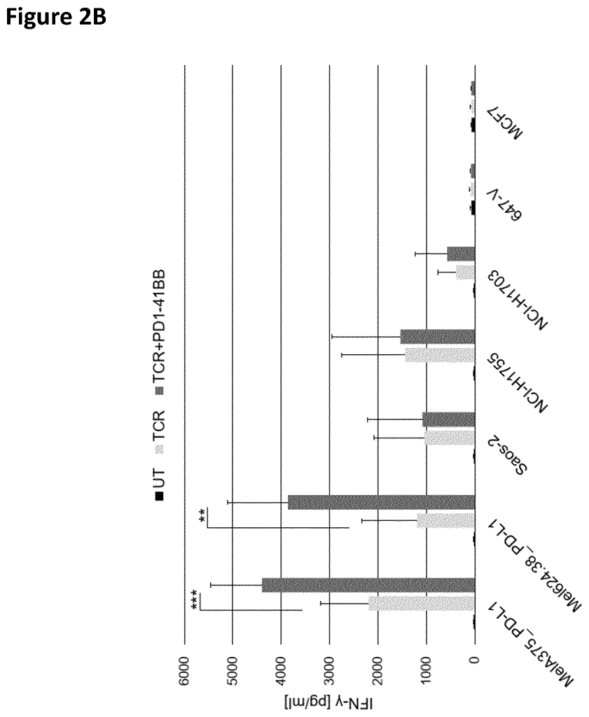

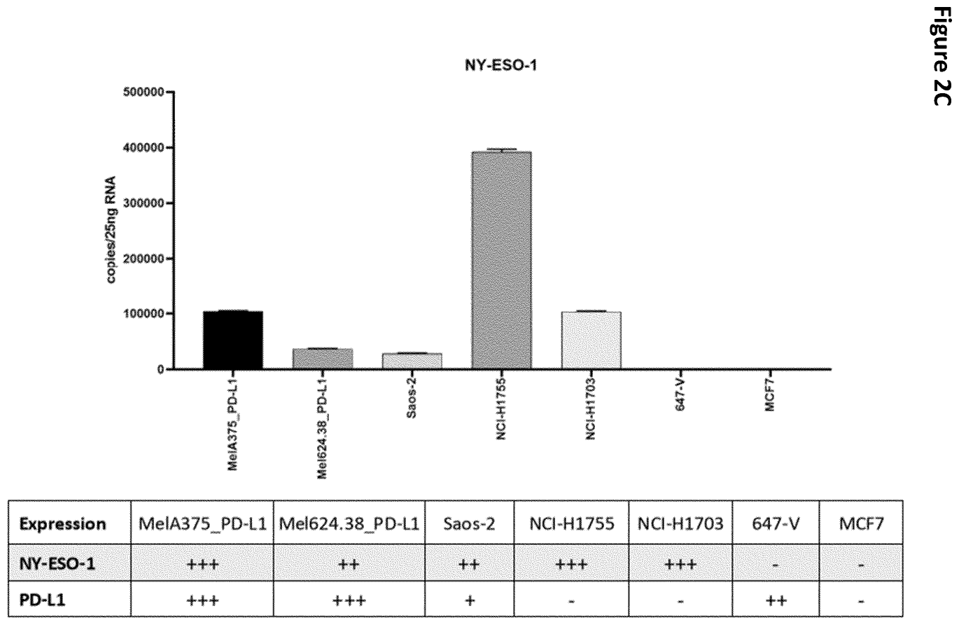

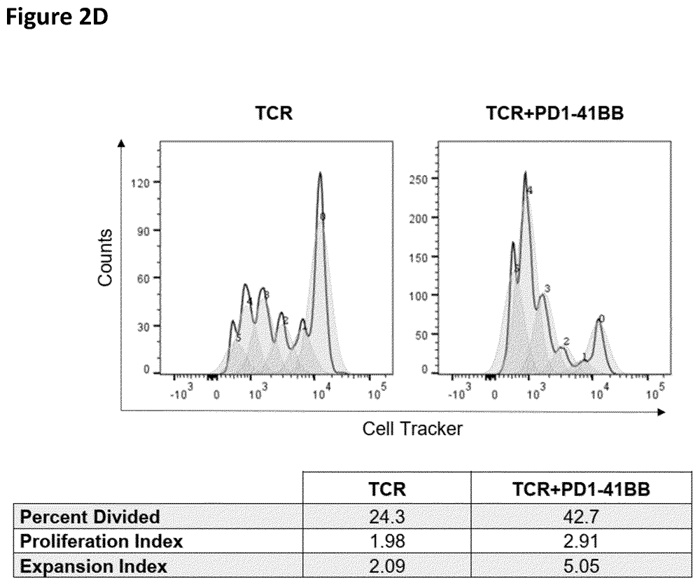

Flow cytometric analysis of untransduced (UT), NY-ESO-1 TCR expressing (TCR) and NY-ESO-1 TCR co-expressing PD1-41BB (TCR+PD1-41BB) T cells was performed (FIG. 2A). NY-ESO-1 TCR transgenic CD8+ T cells with or without PD1-41BB were cultured with tumor cell lines expressing different levels of NY-ESO-1 and PD-L1 and released IFN-γ levels were determined by ELISA (t-test, **p>0.01, ***p<0.001) (FIG. 2B). Expression levels of NY-ESO-1 were measured by qPCR and quantified against a standard calibration curve. The levels of PD-L1 summarized here were evaluated by both qPCR and FACS analysis (FIG. 2C). NY-ESO-1 TCR transgenic CD8+ T cells with or without PD1-41BB were stained with the dye membrane tracker and co-cultured with NY-ESO-1+PD-L1+ tumor cell lines for 5 days. Proliferation was analyzed via flow cytometry and data were evaluated using FlowJo (FIG. 2d).

### Example 3: Enhanced NY-ESO-1 TCR-T Cell Polyfunctionality by Co-Expression of Chimeric Co-Stimulatory Receptor PD1-41BB

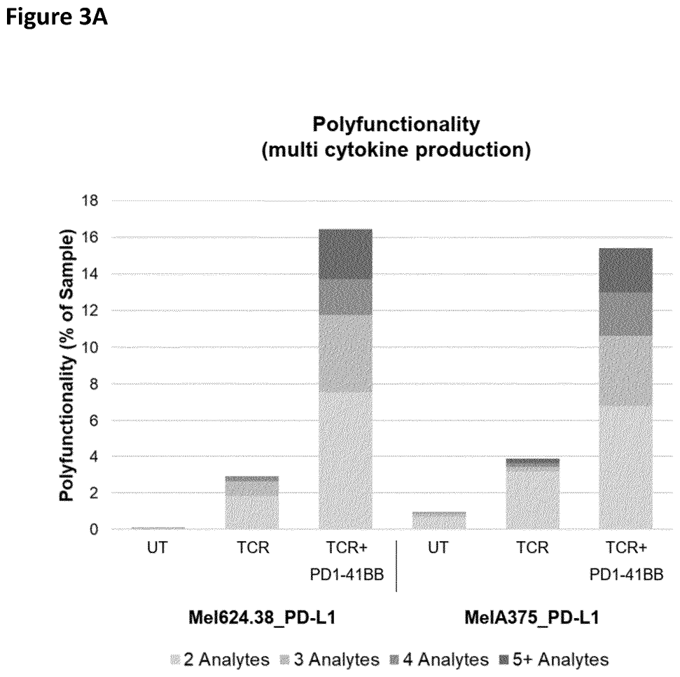

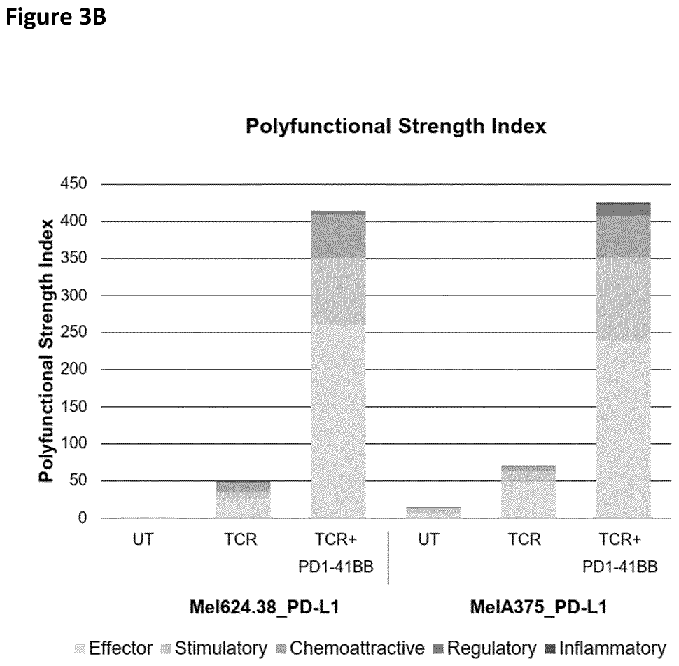

NY-ESO-1 specific TCR-T cells with (TCR+PD1-41BB) and without (TCR) co-expression of chimeric co-stimulatory receptor PD1-41BB were analyzed after 24 hours of co-culture with tumor target cell lines Mel624.38_PD-L1 and MelA375_PD-L1 using single cell proteomic analysis of a panel of 32 secreted cytokines, chemokines, and cytotoxic molecules (IsoLight technology, IsoPlexis). Co-culture with un-transduced T cells (UT) of the same donor served as control. Cells secreting 2 or more cytokines are considered polyfunctional. Polyfunctionality of single TCR T cells displayed as % of sample and categorized in % cells expressing 2, 3, 4 and 5+ analytes (shades of grey) at the same time (FIG. 3 A). Polyfunctional Strength Index (PSI) of the displayed samples is defined as the number of T cells secreting more than 2 effector molecules per cell (polyfunctional T cells in A)), multiplied by mean fluorescence intensity (MFI) of the proteins secreted by the respective cells and categorized in proteins associated with effector, stimulatory, chemoattractive, regulatory and inflammatory properties (shades of grey) (FIG. 3B). Single-cell polyfunctional heat map displays the single-cell cytokine combinations secreted by each sample. Each column represents a specific combination of cytokines, whereas the orange squares depict the frequency at which the specific combination of cytokines is secreted by the corresponding sample (average for target cells Mel624.38_PD-L1 and MelA375_PD-L1 is shown) (FIG. 3C).

### Example 4: Increased Frequencies of Secreted Effector, Stimulatory, and Chemoattractive Proteins by TCR-T Cells Co-Expressing Co-Stimulatory Receptor PD1-41BB

Secretion frequency of single proteins induced by co-culture of TCR T cells equipped with PD1-41BB cells and target cell line Mel624.38_PD-L1. Proteins are displayed and categorized by function due to their a) effector, b) stimulatory, c) chemoattractive, d) regulatory and e) inflammatory properties (FIG. 4).

### Example 5: Serial Killing of Tumor Cell Spheroids

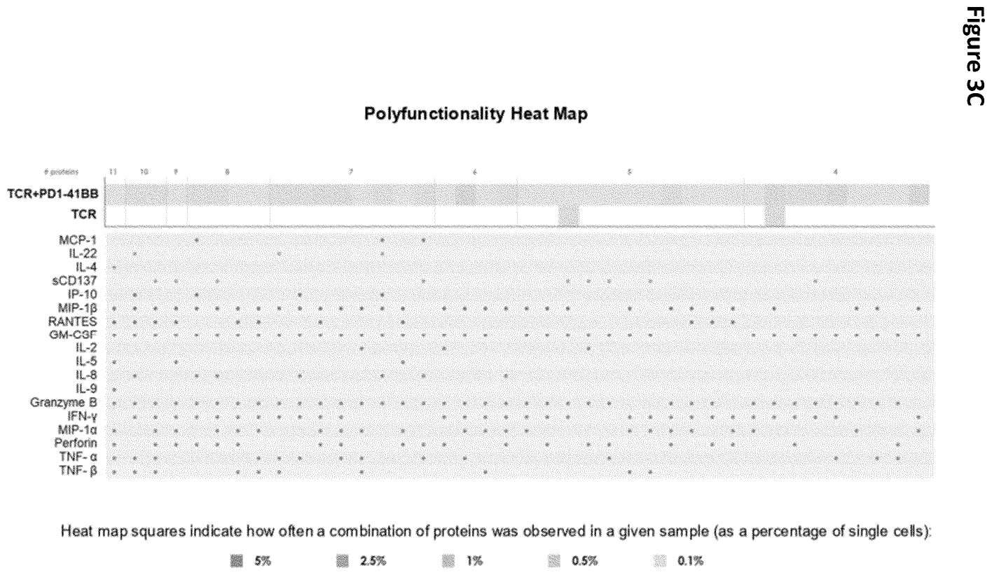

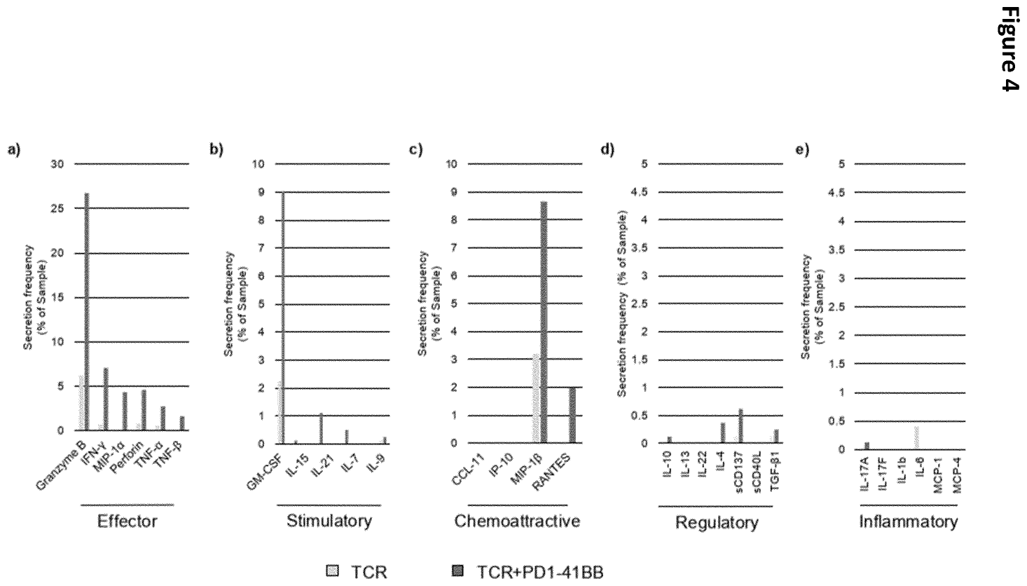

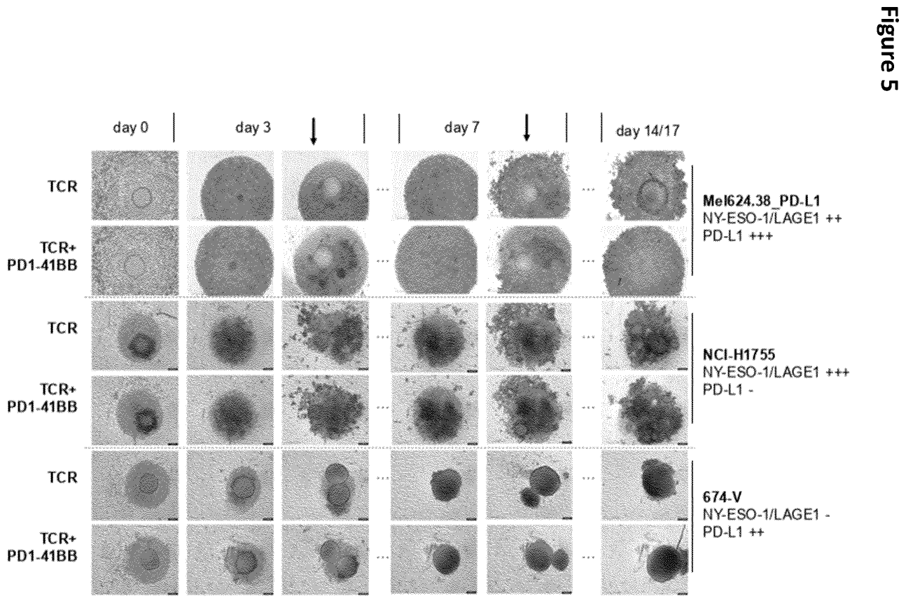

Tumor cell spheroids were generated starting with 1000 NucLightRed-transduced cells in 96 well ULA plates 72 h before co-culture. TCR-transduced T cells with and without PD1-41BB (10,000-38,000 per well) were added and tumor cell killing was monitored by real-time imaging of NucLightRed signal using the IncuCyte S3. Fresh tumor cell spheroids, generated as described above, were added every 72-96 h (FIG. 5).

The application further comprises the following items:

Item 1. A cell population comprising cells expressing
(A) an antigen specific TCR, and
(B) a chimeric co-stimulatory receptor,
wherein the cell population comprises cells which secrete at least two proteins.

Item 2. A cell population according to item 2, wherein the chimeric co-stimulatory receptor comprises
an extracellular domain containing a polypeptide derived from PD-1,
a transmembrane domain, and
an intracellular domain containing a polypeptide derived from 4-1BB,

wherein the cell population comprises cells which secrete at least two proteins.

Item 3. A cell population according to item 2, wherein the chimeric co-stimulatory receptor comprises
an extracellular domain containing a polypeptide derived from PD-1,
a transmembrane domain is derived from PD-1, and
an intracellular domain containing a polypeptide derived from 4-1BB,

wherein the cell population comprises cells which secrete at least two proteins.

Item 4. A cell population according to item 3, comprising cells expressing
(A) a NY-ESO-1/LAGE-1 specific TCR, comprising
   a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
   a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40; and
(B) a chimeric co-stimulatory receptor comprising
   an extracellular domain containing a polypeptide derived from PD-1,
   a transmembrane domain, and
   an intracellular domain containing a polypeptide derived from 4-1BB,

wherein the cell population comprises cells which secrete at least two proteins.

Item 5. The cell population according to any one of the preceding items, wherein the cell population comprises cells which secrete at least three proteins.

Item 6. The cell population according to anyone of the preceding items, wherein the cell population comprises cells which secret at least four proteins.

Item 7. The cell population according to anyone of the preceding items, wherein the cell population comprises cells which secret at least five proteins.

Item 8. The cell population according to anyone of the preceding items, wherein the proteins are selected from the group of effector proteins, stimulatory cytokines and chemo-attractive cytokines.

Item 9. The cell population according to item 8, wherein the effector proteins are selected from the group consisting of Gzm-B, IFN-γ, Perforin, TNF-α, TNF-β, MIP-1α.

Item 10. The cell population according to item 9, wherein the effector proteins are selected from the group consisting of Gzm-B, IFN-γ, Perforin, TNF-α, TNF-β.

Item 11. The cell population according to item 8 to 9, wherein the stimulatory cytokines are selected from the group consisting of GM-CSF, IL-2, IL-5, IL-7, IL-8, IL-9, IL-12.

Item 12. The cell population according to item 8 to 11, wherein the stimulatory cytokines are selected from the group consisting of GM-CSF, IL-2, IL-7, IL-8, IL-9, IL-12.

Item 13. The cell population according to items 8 to 12, wherein the chemo-attractive cytokines are selected from IP-10 and MIP-1β.

Item 14. The cell population according to any one of the preceding items, wherein the proteins are selected from the group consisting of Gzm-B, IFN-γ, Perforin, TNF-α, TNF-β, MIP-1α, GM-CSF, IL-2, IL-5, IL-7, IL-8, IL-9, IL-12, IP-10 and MIP-1β.

Item 15. The cell population according to any one of the preceding items, wherein the proteins are selected from the group consisting of Gzm-B, IFN-γ, Perforin, TNF-α, TNF-β GM-CSF, IL-2, IL-8, MIP-1β and IP-10.

Item 16. The cell population according to any one of the preceding items, wherein the proteins are selected from the group consisting of Gzm-B, IFN-γ, Perforin, TNF-α, TNF-β GM-CSF, IL-2, MIP-1β.

Item 17. The cell population according to any one of the preceding items, wherein the proteins are selected from the group consisting of IFN-γ, Gzm-B, and IP-10.

Item 18. The cell population according to any one of the preceding items, wherein the proteins are selected from the group consisting of IFN-γ and Gzm-B.

Item 19. The cell population according to any one of items 4 and 6 to 18, wherein the TCR is capable of binding to NY-ESO peptide having the amino acid sequence set out in SEQ ID NO: 34 or a portion thereof, preferably its HLA-A2 bound form.

Item 20. The cell population according to item 19, wherein the HLA-A2 is an HLA-A*02:01, HLA-A*02:02, HLA-A*02:04 or HLA-A*02:09 encoded molecule preferably HLA-A*2:01 encoded molecule.

Item 21. The cell population according to anyone of item 3 to 20, wherein the extracellular domain containing a polypeptide derived from PD-1 comprises the sequence of SEQ ID NO: 28 and

wherein the intracellular domain containing a polypeptide derived from 4-1BB comprises the sequence of SEQ ID NO: 32.

Item 22. The cell population according to anyone of items 3 to 21, wherein the transmembrane domain is derived from PD-1, wherein preferably the transmembrane domain containing a polypeptide derived from PD-1 comprises the sequence of SEQ ID NO: 30, preferably wherein the chimeric co-stimulatory receptor comprises the sequence of SEQ ID NO: 26.

Item 23. The cell population according to anyone of the preceding items for use in the treatment of cancer.

Item 24. A target specific immune cell expressing
(A) an antigen specific TCR, and
(B) a chimeric co-stimulatory receptor,

wherein the target specific immune cell secretes at least two proteins.

Item 25. The target specific immune cell according to item 24, wherein the chimeric co-stimulatory receptor comprises
an extracellular domain containing a polypeptide derived from PD-1,
a transmembrane domain, and
an intracellular domain containing a polypeptide derived from 4-1BB,

wherein the target specific immune cell secretes at least two proteins.

Item 26. The target specific immune cell according to item 25, wherein the chimeric co-stimulatory receptor comprises
an extracellular domain containing a polypeptide derived from PD-1,
a transmembrane domain is derived from PD-1, and
an intracellular domain containing a polypeptide derived from 4-1BB,

wherein the target specific immune cell secretes at least two proteins.

Item 27. The target specific immune cell according to any one of items 24 to 26, wherein the target specific immune cell expresses
(A) a NY-ESO-1/LAGE-1 specific TCR, comprising
   a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
   a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40;
(B) a chimeric co-stimulatory receptor comprising
   an extracellular domain containing a polypeptide derived from PD-1,
   a transmembrane domain, and
   an intracellular domain containing a polypeptide derived from 4-1BB,

wherein the target specific immune cell secretes at least two proteins.

Item 28. The target specific immune cell according to any one of items 24 to 27, wherein the target specific immune cell secretes at least three proteins.

Item 29. The target specific immune cell according to anyone of items 24 to 28, wherein the target specific immune cell secretes at least four proteins.

Item 30. The target specific immune cell according to anyone of items 24 to 29, wherein the target specific immune cell secretes at least five proteins.

Item 31. The target specific immune cell according to anyone of items 24 to 30, wherein the proteins are selected from the group of effector proteins, stimulatory cytokines and chemo-attractive cytokines.

Item 32. The target specific immune cell according to item 31, wherein the effector proteins are selected from the group consisting of Gzm-B, IFN-γ, Perforin, TNF-α, TNF-β, MIP-1α.

Item 33. The target specific immune cell according to item 32, wherein the effector proteins are selected from the group consisting of Gzm-B, IFN-γ, Perforin, TNF-α, TNF-β.

Item 34. The target specific immune cell according to anyone of items 31 to 33, wherein the stimulatory cytokines are selected from the group consisting of GM-CSF, IL-2, IL-5, IL-7, IL-8, IL-9, IL-12.

Item 35. The target specific immune cell according to anyone of items 31 to 34, wherein the stimulatory cytokines are selected from the group consisting of GM-CSF, IL-2, IL-7, IL-8, IL-9, IL-12.

Item 36. The target specific immune cell according to anyone of items 31 to 35, wherein the chemo-attractive cytokines are selected from IP-10 and MIP-13.

Item 37. The target specific immune cell according to any one of items 24 to 36, wherein the proteins are selected from the group consisting of Gzm-B, IFN-γ, Perforin, TNF-α, TNF-β, MIP-1α, GM-CSF, IL-2, IL-5, IL-7, IL-8, IL-9, IL-12, IP-10 and MIP-1β.

Item 38. The target specific immune cell according to according to anyone of items 24 to 37, wherein the proteins are selected from the group consisting of Gzm-B, IFN-γ, Perforin, TNF-α, TNF-β GM-CSF, IL-2, IL-8, MIP-1β and IP-10.

Item 39. The cell population according to any one of items 24 to 38, wherein the proteins are selected from the group consisting of Gzm-B, IFN-γ, Perforin, TNF-α, TNF-β GM-CSF, IL-2, MIP-1β.

Item 40. The target specific immune cell according to any one of items 24 to 39, wherein the proteins are selected from the group consisting of IFN-γ, Gzm-B, and IP-10.

Item 41. The target specific immune cell according to any one of items 24 to 40, wherein the proteins are selected from the group consisting of of IFN-γ and Gzm-B.

Item 42. The target specific immune cell according to any one of items 27 and 28 to 41, wherein the TCR is capable of binding to NY-ESO peptide having the amino acid sequence set out in SEQ ID NO: 34 or a portion thereof, preferably its HLA-A2 bound form.

Item 43. The target specific immune cell according to item 42, wherein the HLA-A2 is an HLA-A*02:01, HLA-A*02:02, HLA-A*02:04 or HLA-A*02:09 encoded molecule.

Item 44. The target specific immune cell according to item 43, wherein the HLA-A2 is an HLA-A*2:01 encoded molecule.

Item 45. The target specific immune cell according to anyone of item 25 to 43, wherein the extracellular domain containing a polypeptide derived from PD-1 comprises the sequence of SEQ ID NO: 28 and

wherein the intracellular domain containing a polypeptide derived from 4-1BB comprises the sequence of SEQ ID NO: 32.

Item 46. The target specific immune cell according to anyone of items 26 to 45 wherein the transmembrane domain is derived from PD-1, wherein preferably the transmembrane domain containing a polypeptide derived from PD-1 comprises the sequence of SEQ ID NO: 30, preferably wherein the chimeric co-stimulatory receptor comprises the sequence of SEQ ID NO: 26.

Item 47. The target specific immune cell according to anyone of the items 24 to 46, wherein the target specific immune cell is a lymphocyte.

Item 48. The target specific immune cell according to anyone of the items 24 to 47 for use in the treatment of cancer.

1. The target specific immune cell, wherein the target specific immune cell expresses
(A) a NY-ESO-1/LAGE-1 specific TCR, comprising
a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40;
(B) a chimeric co-stimulatory receptor comprising
an extracellular domain containing a polypeptide derived from PD-1,
a transmembrane domain, and
an intracellular domain containing a polypeptide derived from 4-1BB.

2. The target specific immune cell according to claim 2, wherein the chimeric co-stimulatory receptor comprises
an extracellular domain containing a extracellular domain derived from PD-1,
a transmembrane domain derived from PD-1, and
an intracellular domain containing a intracellular domain derived from 4-1BB.

3. The target specific immune cell according to claim 3, wherein the extracellular domain derived from PD-1 comprises an amino acid sequence with up to 0, 1, 2, 3, 4, 5, 6, 7, 8, 9, or 10 amino acid substitutions compared to the extracellular domain derived from PD-1 as set out in SEQ ID NO: 28.

4. The target specific immune cell according to any one of the preceding claims, wherein the intracellular domain derived from 4-1BB comprises an amino acid sequence with up to 0, 1, 2, 3, 4, 5, 6, 7, 8, 9, or 10 amino acid substitutions compared to the intercellular domain derived from 4-1BB as set out in SEQ ID NO: 32.

5. The target specific immune cell according to any one of the preceding claims, wherein the extracellular domain derived from PD-1 comprises the sequence of SEQ ID NO: 28 and wherein the intracellular domain derived from 4-1BB comprises the sequence of SEQ ID NO: 32.

6. The target specific immune cell according to any one of claims 2 to 5, wherein the transmembrane domain derived from PD-1 comprises an amino acid sequence with up to 0, 1, 2, 3, 4, 5, 6, 7, 8, 9, or 10 amino acid substitutions compared to the transmembrane sequence derived from PD-1 as set out in SEQ ID NO: 30.

7. The target specific immune cell according to claim 6, wherein the transmembrane domain is derived from PD-1, wherein preferably the transmembrane domain containing a polypeptide derived from PD-1 comprises the sequence of SEQ ID NO: 30.

8. The target specific immune cell according to any one of the preceding claims, preferably wherein the chimeric co-stimulatory receptor comprises the sequence of SEQ ID NO: 26.

9. The target specific immune cell according to any one of the preceding claims, wherein the target specific immune cell is a lymphocyte.

10. The target specific immune cell according to any one of the preceding claims, wherein the target specific immune cell is a T cell.

11. The target specific immune cell according to anyone of the preceding claims, wherein the TCR comprises a variable TCR α region having an amino acid sequence which is at least 80% identical to SEQ ID NO: 41 and a variable TCR β region having an amino acid sequence which is at least 80% identical to SEQ ID NO: 42.

12. The target specific immune cell according to anyone of the preceding claims, wherein the TCR comprises a variable TCR α region having the amino acid sequence of SEQ ID NO: 41 and a variable TCR β region having the amino acid sequence of SEQ ID NO: 42.

13. The target specific immune cell according to anyone of the preceding claims, wherein the TCR is capable of binding to a NY-ESO peptide having the amino acid sequence set out in SEQ ID NO: 34 or a portion thereof, preferably its HLA-A2 bound form.

14. The target specific immune cell according to anyone of the preceding claims, wherein the HLA-A2 is an HLA-A*02:01, HLA-A*02:02, HLA-A*02:04 or HLA-A*02:09 encoded molecule preferably HLA-A*2:01 encoded molecule.

15. Cell population comprising the target specific immune cells according to claims 1 to 14.

16. The cell population according to claim 15,
wherein the cell population comprises cells which secrete at least two proteins.

17. The cell population according to claim 16, wherein the cell population comprises cells which secrete at least three proteins.

18. The cell population according to claim 16 or 17, wherein the cell population comprises cells which secret at least six proteins.

19. The cell population according to anyone of claims 16 to 18, wherein the proteins are selected from the group of effector proteins, stimulatory cytokines and chemo-attractive cytokines.

20. The cell population according to anyone of claims 16 to 19, wherein at least 5% of the population secrete more than 1 effector proteins,

21. The cell population according to anyone of claims 16 to 20, wherein at least 4% of the population secrete more than one from selected from effector proteins, stimulatory cytokines and chemo attractive cytokines.

22. The cell population according to anyone of claims 16 to 21, wherein the cell secretes of at least one of the proteins selected from RANTES, MIP-1alpha, Perforin, TNF-alpha and TNF-beta.

23. The cell population according to claim 19 to 22, wherein the effector proteins are selected from the group consisting of Gzm-B, IFN-γ, Perforin, TNF-α, TNF-β, MIP-1α.

24. The cell population according to claim 19 to 23, wherein the effector proteins are selected from the group consisting of Gzm-B, IFN-γ, Perforin, TNF-α, TNF-β.

25. The cell population according to claim 19 to 24, wherein the stimulatory cytokines are selected from the group consisting of GM-CSF, IL-2, IL-5, IL-7, IL-8, IL-9, IL-12.

26. The cell population according to claim 19 to 25, wherein the stimulatory cytokines are selected from the group consisting of GM-CSF, IL-2, IL-7, IL-8, IL-9, IL-12.

27. The cell population according to claims 19 to 26, wherein the chemo-attractive cytokines are selected from IP-10 and MIP-1β.

28. The cell population according to claims 16 to 27, wherein the proteins are selected from the group consisting of Gzm-B, IFN-γ, Perforin, TNF-α, TNF-β, MIP-1α, GM-CSF, IL-2, IL-5, IL-7, IL-8, IL-9, IL-12, IP-10 and MIP-1β.

29. The cell population according to claims 16 to 28, wherein the proteins are selected from the group consisting of Gzm-B, IFN-γ, Perforin, TNF-α, TNF-β GM-CSF, IL-2, IL-8, MIP-1β and IP-10.

30. The cell population according to any one of claims 16 to 29, wherein the proteins are selected from the group consisting of Gzm-B, IFN-γ, Perforin, TNF-α, TNF-β GM-CSF, IL-2, MIP-1β.

31. The cell population according to any one of claims 16 to 30, wherein the proteins are selected from the group consisting of IFN-γ, Gzm-B, and IP-10.

32. The cell population according to any one of claims 16 to 31, wherein the proteins are selected from the group consisting of IFN-γ and Gzm-B.

33. Pharmaceutical composition comprising the target specific immune cell as defined in claims 1 to 14 or a cell population as defined in claims 15 to 32.

34. Method of treating cancer in a human or non-human animal in need thereof, comprising administering to said human or non-human animal the target specific immune cell according to claim 1 to 14, or the cell population according to claims 15 to 33.

35. The method of claim 34 wherein the cancer is a solid tumor.


SEQUENCE LISTING (45 sequences)

SEQ ID NO: 1-25 - skipped (25 entries)

SEQ ID NO: 26 - protein, 233 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIKRGRKKLLYIFKQPFMRPVQTTQEEDGCSCRFPEEEEGGCEL

SEQ ID NO: 27 - DNA, 699 bp, synthetic construct
  source 1..699: mol_type = other DNA
atgcaaattcctcaagctccttggcctgtcgtgtgggccgttctgcaacttggatggcggcctggctggttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggactgctgggatctctggtgctgcttgtttgggtgctcgccgtgatcaagcggggcagaaagaagctgctgtacatcttcaagcagcccttcatgcggcccgtgcagaccacacaagaggaagatggctgctcctgcagattccccgaggaagaagaaggcggctgcgaactc

SEQ ID NO: 28 - protein, 170 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 29 - DNA, 510 bp, synthetic construct
  source 1..510: mol_type = other DNA
atgcaaattcctcaagctccttggcctgtcgtgtgggccgttctgcaacttggatggcggcctggctggttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtc

SEQ ID NO: 30 - protein, 21 aa, Homo sapiens
VGVVGGLLGSLVLLVWVLAVI

SEQ ID NO: 31 - DNA, 63 bp, synthetic construct
  source 1..63: mol_type = other DNA
gtgggagttgttggcggactgctgggatctctggtgctgcttgtttgggtgctcgccgtgatc

SEQ ID NO: 32 - protein, 42 aa, Homo sapiens
KRGRKKLLYIFKQPFMRPVQTTQEEDGCSCRFPEEEEGGCEL

SEQ ID NO: 33 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgaactc

SEQ ID NO: 34 - protein, 9 aa, Homo sapiens
SLLMWITQC

SEQ ID NO: 35 - protein, 6 aa, Homo sapiens
DRGSQS

SEQ ID NO: 36 - protein, 6 aa, Homo sapiens
IYSNGD

SEQ ID NO: 37 - protein, 9 aa, Homo sapiens
CAADDKIIF

SEQ ID NO: 38 - protein, 5 aa, Homo sapiens
SGHDY

SEQ ID NO: 39 - protein, 6 aa, Homo sapiens
FNNNVP

SEQ ID NO: 40 - protein, 10 aa, Homo sapiens
CASSRGGSGY

SEQ ID NO: 41 - protein, 129 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPN

SEQ ID NO: 42 - protein, 132 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVE

SEQ ID NO: 43 - protein, 269 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 44 - protein, 308 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 45 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR